(19) United States
(12) Patent Application Publication
(10) Pub. No.: US 2015/0018873 A1
(43) Pub. Date: Jan. 15, 2015

(54) BALLOON CATHETER

(71) Applicant: TERUMO KABUSHIKI KAISHA, TOKYO (JP)

(72) Inventor: Taiga Nakano, Kanagawa (JP)

(21) Appl. No.: 14/383,980

(22) PCT Filed: Mar. 1, 2013
(86) PCT No.: PCT/JP2013/055736

§ 371 Date: Sep. 9, 2014

(30) Foreign Application Priority Data
Mar. 9, 2012 (JP) 2012-053750

(51) Int. Cl. A61B 17/12; A61M 25/10; A61B 17/00; A61B 17/24

(52) U.S. Cl. CPC A61B 17/12031; A61B 17/12104; A61B 17/12136; A61B 17/24; A61M 25/1011; A61B 17/00491; A61B 2017/00809; A61M 2025/1013; A61M 2025/1086
USPC 606/215

(57) ABSTRACT

To provide a balloon catheter that enables a minimally invasive and effective pulmonary emphysema treatment. The balloon catheter includes: a first balloon that has at least a part of an external surface thereof come into contact with a biological tissue when expanded in living body; a second balloon expandable and contractible separately from the first balloon; a main body portion having a first lumen through which a fluid for expanding the first balloon can circulate and a second lumen through which a fluid for expanding the second balloon can circulate; and supply portions that supply the first balloon with an adhesive material for adhering the external surface of the first balloon to the biological tissue.

### CROSS REFERENCE TO RELATED APPLICATIONS

This application is based on International Application No. PCT/JP2013/055736 filed on Mar. 1, 2013, which claims priority to Japanese National Application No. 2012-053750 filed on Mar. 9, 2012. The entire contents of each and every foregoing application are incorporated herein by reference.

### BACKGROUND

The present invention relates to a balloon catheter used for the treatment of pulmonary emphysema.

Pulmonary emphysema is known as one of chronic obstructive pulmonary diseases. Pulmonary emphysema is a disease in which septa of pulmonary alveoli that exchange oxygen and carbon dioxide are broken, whereby the pulmonary alveoli are connected to each other, the broken pulmonary alveoli bulge greatly into a state without elasticity or contractility (fibrosing state), and the effective area of the pulmonary alveoli is decreased, thus lowering a ventilation capability.

Normal pulmonary alveoli are contracted as the lung is contracted in exhalation. However, when pulmonary emphysema is developed, a symptom appears in which the pulmonary alveoli are not contracted in exhalation, bronchi are squeezed by surrounding pulmonary alveoli that cannot be contracted when the lung is contracted, and thus an air does not come out easily. A treatment method using an airway bypass as shown in JP-T-2003506132, for example, has been proposed as a method for the treatment of such pulmonary emphysema. In a treatment using an airway bypass, an opening portion is formed in an airway wall, and a bypass to adjacent enlarged pulmonary alveoli is formed. Therefore, any exhaled air is not passed through a narrowed bronchus, but the exhaled air is allowed to escape directly into the airway. When such a bypass is to be formed, a puncture tool is used to reach a site where an opening in the airway is to be formed. A puncture is performed and a hole in the airway wall which hole is formed via the aforementioned puncture is expanded by a balloon. Finally a member for maintaining the opened state is left indwelling in the opening portion so that the opening is not closed after healing. However, in such a treatment, the opening portion is formed by puncturing the airway wall. Thus, the procedure involves bleeding, which puts a heavy burden on a living body.

On the other hand, Japanese Patent Laid-Open No. 2009-297548 and U.S. Pat. No. 6,682,520 propose methods for decreasing a volume of emphysematous pulmonary alveoli to recover a ventilation capability without performing a surgical procedure as described above.

The method described in Japanese Patent Laid-Open No. 2009- uses an obturator for closing a bronchus to block the supply of an air, and thereby decreases the volume of diseased pulmonary alveoli. On the other hand, the method described in Patent Document 3 administers a gel for promoting the progress of a fibrosis to collapse a biological tissue forming diseased pulmonary alveoli, and thereby decreases the volume of the pulmonary alveoli. Accordingly minimally invasive treatments, as compared with the treatment method using the airway bypass, can be realized.

### SUMMARY OF THE DISCLOSURE

When the obturator is used, the obturator left indwelling in the bronchus may fall off, or may be positionally displaced from the indwelling position. Therefore, a reliable treatment effect cannot be expected. In addition, when the gel for prompting a fibrosis is used, the treatment effect varies from patient to patient. Thus, some patients may not be able to obtain a sufficient treatment effect.

The present disclosure has been made in order to solve the above problems. It is an intention of the present disclosure to provide a balloon catheter that enables a minimally invasive and effective pulmonary emphysema treatment.

A balloon catheter according to the present disclosure for achieving the above intention has the following constitution.

A balloon catheter in accordance with the instant disclosure includes: a balloon provided so as to be expandable and contractible such that at least a part of an external surface of the balloon is brought into contact with a biological tissue when the balloon is expanded in living body. The balloon has a main body portion having a lumen through which a fluid for expanding the balloon can circulate, and a supply portion for supplying the external surface of the balloon with an adhesive material having fluidity. The adhesive material adheres the external surface of the balloon to the biological tissue.

The balloon catheter is provided so as to be expandable by injection of the adhesive material supplied through the lumen.

The supply portion of the balloon catheter has a through hole penetrating from an inside of the balloon adhered to the biological tissue to the external surface of the balloon.

Further, the supply portion has a discharge portion provided in the main body portion, the discharge portion discharging the adhesive material to the external surface of the balloon adhered to the biological tissue.

At least a part of the external surface of the balloon is formed so as to have higher compliance than other parts in the external surface.

The aforementioned balloon includes a first balloon having the external surface brought into contact with the biological tissue. This first balloon is expanded by injection of the adhesive material, and a second balloon disposed such that at least a part of the second balloon is covered by the first balloon The second balloon being expandable and contractible separately from the first balloon The main body portion of the above-described balloon includes a first lumen for circulating the adhesive material to and from the first balloon, and a second lumen for circulating a fluid for expanding the second balloon to and from the second balloon.

The balloon catheter further includes a connecting portion for connecting the first balloon and the second balloon to each other.

The balloon catheter wherein compliance of the first balloon is higher than compliance of the second balloon.

The external surface of the balloon adhered to the biological tissue is provided with a guide portion for guiding a flow of the adhesive material, and

The balloon catheter wherein the external surface of the balloon adhered to the biological tissue is provided with a projecting portion projecting toward the biological tissue.

The balloon catheter in accordance with the instant disclosure further includes an attaching portion for separably attaching the balloon adhered to the biological tissue to the main body portion.

According to the disclosure provided herein-above, after the balloon provided to the balloon catheter is expanded in living body, the adhesive material having fluidity is supplied to the external surface of the balloon to adhere the external surface of the balloon to the biological tissue of diseased pulmonary parenchyma. Further, the balloon is contracted. Therefore, the pulmonary parenchyma can be contracted as the balloon is contracted. As a result, the volume of the diseased pulmonary parenchyma can be decreased. According to the procedure using the balloon catheter, the pulmonary parenchyma can be physically contracted as the balloon is contracted. Thus, the puncturing operation performed in the bypass technique is rendered unnecessary. A minimally invasive procedure can therefore be realized. Further, no variations in treatment effect occur each time the procedure is performed and thus a stable treatment effect can therefore be obtained.

When the balloon is provided so as to be expandable by the injection of the adhesive material supplied through the lumen provided in the main body portion, the expansion of the balloon and the adhesion of the external surface of the balloon to the biological tissue can be performed simultaneously. Thus, the procedure using the balloon catheter can be performed more simply and more quickly.

In another embodiment, when the balloon catheter has the through holes that penetrate from the inside of the balloon to the external surface of the balloon adhesive material flowing into the inside of the balloon can be made to flow out to the external surface of the balloon via the through holes. Thus, the adhesiveness of the external surface of the balloon to the biological tissue can be enhanced.

In another embodiment, when the balloon catheter has the discharge portions that discharge the adhesive material to the external surface of the balloon the adhesive material can be directly applied to the external surface of the balloon. Thus, the adhesiveness of the external surface of the balloon to the biological tissue can be enhanced. The effect of treatment with the balloon catheter can therefore be improved.

In an additional embodiment, when at least a part of the external surface of the balloon is formed so as to have higher compliance than other parts in the external surface, the part formed so as to have higher compliance in the external surface can be partially deformed flexibly while the expandability of the balloon as a whole due to the introduction of the fluid is maintained. It is thereby possible to improve the contact property between the external surface of the balloon and the biological tissue, and thus increase the contact area between the external surface of the balloon and the biological tissue. The effect of treatment with the balloon catheter can therefore be improved.

In a further embodiment, when the balloon catheter has the first balloon and the second balloon disposed inside the first balloon the contacting of the first balloon with the biological tissue and the drawing of the biological tissue in the contracting direction of the first balloon can be performed smoothly by controlling the expansion and contraction of the second balloon.

When the balloon catheter has the connecting portions that connect the first balloon and the second balloon to each other, the expansion and contraction following properties of the first balloon following the expansion and contraction of the second balloon can be improved. The effect of treatment with the balloon catheter can therefore be improved.

Furthermore, when the compliance of the second balloon is higher than the compliance of the first balloon, the external surface of the first balloon can be deformed flexibly so as to be along the inner surface shape of the biological tissue when the first balloon is expanded. Thus, the contact area where the external surface of the first balloon is in contact with the biological tissue can be increased. The effect of treatment with the balloon catheter can therefore be improved.

In yet another embodiment, when the external surface of the balloon brought into contact with the biological tissue is provided with the guide portions that guide flows of the adhesive material, the guide portions can distribute the adhesive material over the entire external surface of the balloon. Therefore, the adhesiveness of the external surface of the balloon to the biological tissue can be improved.

In an additional embodiment, when the external surface of the balloon brought into contact with the biological tissue is provided with the projecting portions that project toward the biological tissue the external surface of the first balloon can be brought into close contact with the biological tissue. Thus, the contact area between the external surface of the first balloon and the biological tissue can be increased. The effect of treatment with the balloon catheter can therefore be improved.

In yet an additional embodiment, when the attaching portions that separably attach the balloons to the main body portion are provided, a procedure for leaving the balloons indwelling in the pulmonary parenchyma can be performed. A degree of freedom of the procedure with the balloon catheter can therefore be increased.

### BRIEF DESCRIPTION OF DRAWINGS

FIG. 1 illustrates in a simplified manner, a general constitution of a balloon catheter according to an embodiment of the present disclosure.

FIG. 2 illustrates the parts of the balloon catheter according to the embodiment; FIG. 2(A) being a perspective view showing a balloon provided to the balloon catheter in enlarged dimension, FIG. 2(B) being a sectional view taken along an arrow line 2B-2B of FIG. 1, and FIG. 2(C) being a sectional view taken along an arrow line 2C-2C of FIG. 1.

FIG. 3 illustrates in enlarged dimension, a part indicated by alternate long and short dashed lines 3B in FIG. 2(B).

FIGS. 4(A) to 4(C) illustrate examples of a shape of through holes provided in the balloon catheter.

FIG. 5 facilitates the explanation a method of treatment with the balloon catheter according to the embodiment; FIG. 5(A) being a diagram showing a state in which the balloon provided to the balloon catheter is introduced into a diseased pulmonary alveolus, and FIG. 5(B) being a diagram showing a state in which the balloon is expanded from the state of FIG. 5(A).

FIG. 6 facilitates the explanation of the method of treatment with the balloon catheter according to the embodiment; FIG. 6(A) being a diagram showing a state in which the balloon is contracted from the state of FIG. 5(B), and FIG. 6(B) being a diagram showing a state in which the balloon is further contracted from the state of FIG. 6(A).

FIG. 7 facilitates that explanation of the method of treatment with the balloon catheter according to the embodiment; FIG. 7(A) being a diagram showing a state in which a pulmonary parenchyma adhering to the balloon is drawn into an airway together with the balloon from the state of FIG. 6(B), and FIG. 7(B) being a diagram showing a state in which the balloon is detached from the main body portion of the balloon catheter and the balloon is left indwelling within the airway.

FIG. 8 illustrates explaining modifications of the balloon catheter according to the embodiment.

### DETAILED DESCRIPTION

An embodiment of the present invention will hereinafter be described with reference to the drawings. It is to be noted that dimensional ratios of the drawings are exaggerated for the convenience of description, and may be different from actual ratios.

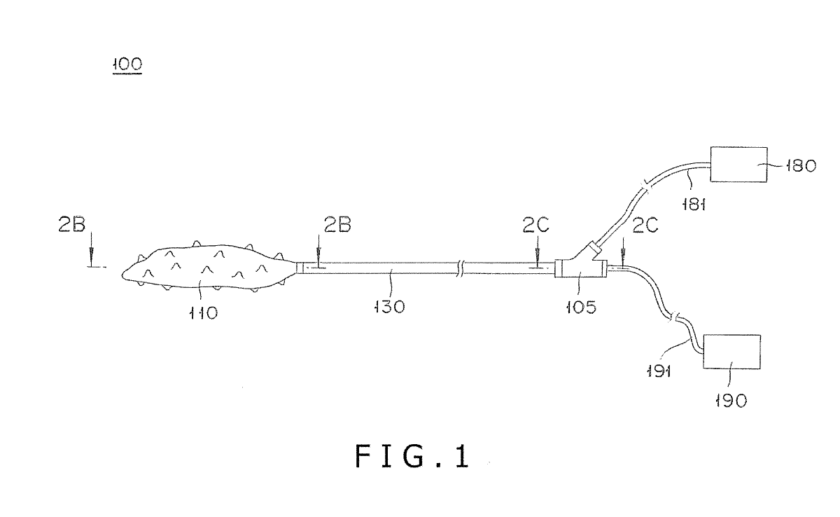

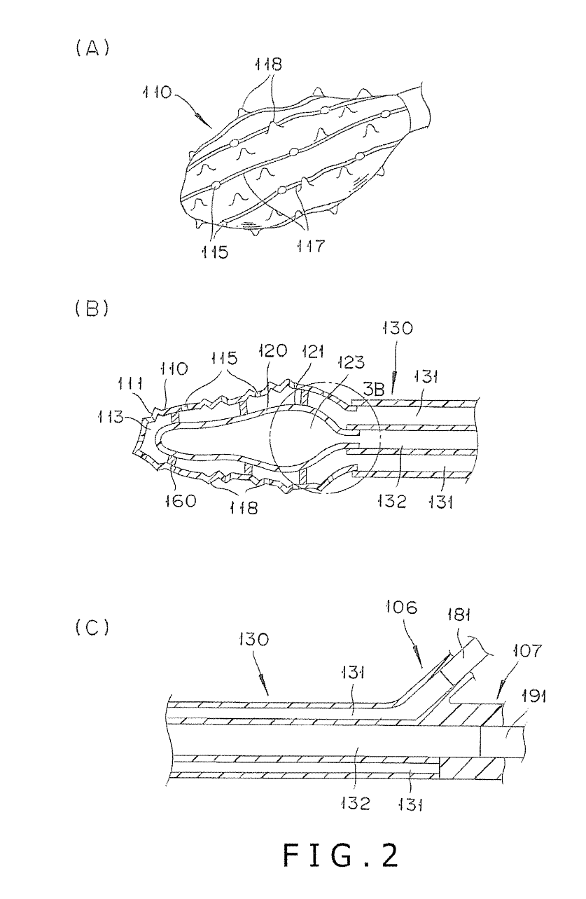

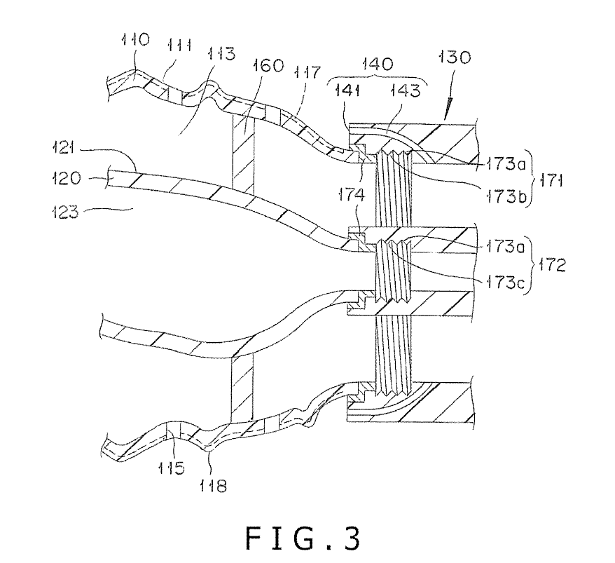

FIG. 1 shows a general constitution of a balloon catheter according to an embodiment of the present invention. FIGS. 2(A) to 2(C) are enlarged views of assistance in explaining parts of the balloon catheter. FIG. 3 is an enlarged view of assistance in explaining principal parts of a balloon provided to the balloon catheter.

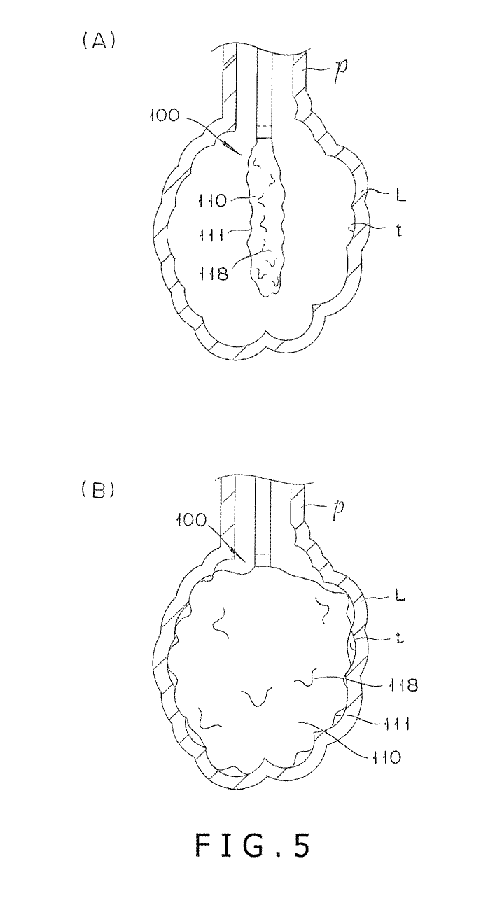

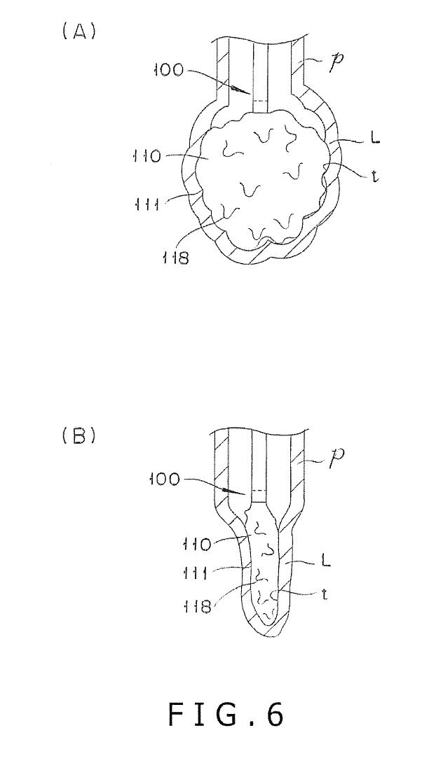

A balloon catheter 100 as shown in FIG. 1 can be used as a medical device for use in treatment of pulmonary emphysema, for example. The balloon catheter 100 decreases a volume of a diseased pulmonary parenchyma L (a respiratory bronchiole, a pulmonary alveolus, an alveolar duct, an alveolar sac, or the like) of a patient with pulmonary emphysema, thereby making in possible to increase an effective area of the pulmonary parenchyma L that contributes to respiration and thus improve ventilation capability. One example of an application of the balloon catheter 100, as shown in FIG. 5 and FIG. 6, a first balloon 110 provided to the balloon catheter 100 is expanded in living body. Thereafter, an external surface 111 of the first balloon 110 is supplied with an adhesive material having fluidity, so that the external surface 111 is adhered to a biological tissue t of the diseased pulmonary parenchyma L. Further, the first balloon 110 is contracted, and the pulmonary parenchyma L is contracted as the first balloon 110 is contracted. Such a procedure can decrease the volume of the diseased pulmonary parenchyma L. The present embodiment will be described below in detail.

As shown in FIG. 1, FIG. 2(B), and FIG. 3, the balloon catheter 100 includes: the first balloon 110 and a second balloon 120 capable of being expanded by injection of a fluid; a long main body portion 130 to which the first balloon 110 and the second balloon 120 are attached; and a hub 105 provided with ports to which various kinds of fluid tubes can be connected.

As shown in FIG. 1, when a treatment using the balloon catheter 100 is performed, an adhesive material supply source 180 and a fluid supply source 190 can be used together with the balloon catheter 100.

The adhesive material supply source 180 is a device for supplying an adhesive material to the balloon catheter 100. A cyanoacrylate-based adhesive, for example, can be used as the adhesive material. The cyanoacrylate-based adhesive includes for example Aron Alpha A for medical use (manufactured by Daiichi Sankyo Company, Limited), Dermabond (manufactured by J&J)®, Histoacryl (manufactured by B. Brown)®, and other aminoacrylate-based adhesives know to those having skill in the art.

Adhesive materials obtained by synthesizing or mixing hydrophobic polymers (monomers) and hydrophilic polymers (monomers), for example, can also be used as the adhesive material. The hydrophobic material includes for example acrylamide and polyethylene. The hydrophilic material includes for example polylysine, chitosan, and other cationic polymers (monomers), carboxymethylcellulose and other anionic polymers (monomers), and polyvinyl alcohol.

A mode can be adopted in which a hydrophilic (cationic polymer) coating is applied to the balloon side in advance and an anionic polymer is applied and used as an adhesive, or a mode can be adopted in which, conversely, an anionic polymer coating is applied to the balloon side in advance and a cationic polymer is applied and used as an adhesive. Further, an adhesive for living bodies, such as isopropylacrylamide (NIPAM) or the like, can be used.

A refrigerant such as a liquid or gas cooled to a predetermined temperature (about −20 to −100° C.), for example, can be used as the adhesive material. Incidentally, the value of the temperature can be changed as long as the first balloon 110 is bonded to the biological tissue t.

The fluid supply source 190 is a device for supplying a fluid as a pressurizing medium for expanding the second balloon 120. As the pressurizing medium, for example a physiological saline solution or another liquid, a gas such as an air or the like, a solid dispersed in a liquid or in a gas, an aggregate of particles, or the like can be used.

As shown in FIG. 2(B) and FIG. 3, the first balloon 110 includes an internal space 113 into which the fluid for expanding the first balloon 110 flows and an external surface 111 at least a part of which is brought into contact with the biological tissue t when the first balloon 110 is expanded (see FIG. 6). The internal space 113 of the first balloon 110 communicates with a first lumen 131 provided in the main body portion 130. The adhesive material is supplied to the first balloon 110 via the first lumen 131. That is, the first balloon 110 can be expanded by the adhesive material supplied through the first lumen 131.

As shown in FIG. 2(C), the first lumen 131 is provided within the main body portion 130, as a flow passage through which the adhesive material can circulate. A base end of the first lumen 131 is provided with a first port 106 to which one end of a fluid tube 181 for supplying the adhesive material is connected liquid-tightly and airtightly. The other end of the fluid tube 181 connected to the first port 106 is connected to the adhesive material supply source 180 liquid-tightly and airtightly (see FIG. 1). Hence, the adhesive material supplied from the adhesive material supply source 180 flows into the internal space 113 of the first balloon 110 via the fluid tube 181 and the first lumen 131, and expands the first balloon 110.

As shown in FIG. 2(B) and FIG. 3, the second balloon 120 is disposed inside the first balloon 110 such that at least a part of the second balloon 120 is covered by the first balloon 110. An internal space 123 of the second balloon 120 communicates with a second lumen 132 provided in the main body portion 130. The second balloon 120 can be expanded by the pressurizing medium supplied through the second lumen 132. The second balloon 120 can also be contracted by discharging the pressurizing medium supplied to the internal space 123 from the internal space 123 through the second lumen 132. Therefore, the second balloon 120 can be expanded and contracted separately from the first balloon 110 by controlling the supply of the pressurizing medium to the second balloon 120 and the discharge of the pressurizing medium from the second balloon 120.

As shown in FIG. 2(C), the second lumen 132 is provided within the main body portion 130, as a flow passage through which the pressurizing medium can circulate. A base end of the second lumen 132 is provided with a second port 107 to which one end of a fluid tube 191 for supplying the pressurizing medium is connected liquid-tightly and airtightly. Another end of the fluid tube 191 connected to the second port 107 is connected to the fluid supply source 190 liquid-tightly and airtightly (see FIG. 1). Hence, the pressurizing medium supplied from the fluid supply source 190 flows into the internal space 123 of the second balloon 120 via the fluid tube 191 and the second lumen 132, and expands the second balloon 120.

The main body portion 130 of the balloon catheter 100 can be formed of, for example, a polymeric material having excellent flexibility, such as polyurethane, polyolefin, polyester, polycarbonate, polysulfone, silicone, or the like.

First and second balloon 110 and 120, respectively may be fabricated from methods known in the medical field for manufacturing balloon catheters. Such materials may include, for example, polyethylene, polypropylene, polybutene, ethylene-propylene copolymers, ethylene-vinyl acetate copolymers, ionomers, and other polyolefins, and crosslinked products or partially crosslinked products thereof, polyethylene terephthalate and other polyesters, polyester elastomers, polyvinyl chloride, polyurethane, polyurethane elastomers, polyphenylene sulfide, polyamide, polyamide elastomers, fluorocarbon resins, and other polymeric materials, silicone rubbers, latex rubbers, and the like. In addition, the materials may be mixtures of two or more of these materials, and include films (or sheets) obtained by laminating these materials as appropriate. In addition, each balloon may be provided with a radiation marker or the like.

A contact property between the biological tissue t and the external surface 111 of the first balloon 110 can be improved by adjusting the compliance of the balloons 110 and 120 of the balloon catheter 100. Incidentally, the compliance in this case will be described as an index for indicating a degree of ease of deformation of balloon shape. The contact property is improved for the following reasons. The external surface 111 of the first balloon 110 is adhered to the biological tissue t of the pulmonary parenchyma L in a state of being in contact with the biological tissue t of the pulmonary parenchyma L. However, in a case where a contact area between the external surface 111 of the first balloon 110 and the biological tissue t is small when the external surface 111 of the first balloon 110 is adhered, a strength of adhesion with which the first balloon 110 adheres to the biological tissue t is decreased. In such a case, it is difficult to decrease the volume of the pulmonary parenchyma L by drawing the biological tissue t in a contracting direction as the balloon is contracted. It is therefore desirable to improve the contact property between the biological tissue t and the external surface 111 of the first balloon 110.

Modes in which the compliance of the balloons is adjusted include for example a mode in which parts different from each other in compliance are provided in the external surface 111 of the first balloon 110. Specifically, at least a part of the external surface 111 of the first balloon 110 is formed so as to have higher compliance than other parts in the external surface 111 of the first balloon 110 (formed so as to change more in balloon shape in response to a change in a pressure for expanding the balloon). This enables the part formed so as to have higher compliance in the external surface 111 to be partially deformed flexibly while the expandability of the first balloon 110 as a whole due to the introduction of the fluid is maintained. It is therefore possible to improve the contact property between the external surface 111 of the first balloon 110 and the biological tissue t, and thus increase the contact area between the external surface 111 of the first balloon 110 and the biological tissue t. Incidentally, the part of high compliance and the part of low compliance in the first balloon 110 can be provided at arbitrary positions according to the external dimensions and the external shape of the first balloon 110 or other convenience in design.

Modes in which the compliance is adjusted include for example a mode in which the compliance of the first balloon 110 and the compliance of the second balloon 120 are made different from each other. Specifically, the compliance of the first balloon 110 is made higher than the compliance of the second balloon 120. When the compliance of the first balloon 110 is increased, the external surface 111 of the first balloon 110 can be deformed flexibly so as to be along the inner surface shape of the biological tissue t when the first balloon 110 is expanded. Therefore, the contact area where the external surface 111 of the first balloon 110 is in contact with the biological tissue t can be increased. In addition, when the second balloon 120 is expanded in a state in which the external surface 111 of the first balloon 110 is adhered to the biological tissue t, the second balloon 120 supports the first balloon 110 from the inside so as to press the first balloon 110 against the biological tissue t. Therefore, the adhesiveness of the external surface 111 of the first balloon 110 to the biological tissue t can be enhanced. However, when the compliance of the second balloon 120 is relatively high, such a supporting function of the second balloon 120 is not exerted easily. That is, it is also possible to improve a supporting force of the second balloon 120 by setting the compliance of the first balloon 110 higher than the compliance of the second balloon 120.

As for the adjustment of the compliance for improving the contact property of the external surface 111 of the first balloon 110, in the case where the balloon catheter 100 is provided with the first balloon 110 and the second balloon 120, the two modes described above may be adopted, or only one of the modes may be adopted. In addition, in a mode in which a balloon catheter is provided with only a first balloon 110 without a second balloon 120 being used, as will be described herein-below, the method of adjusting the compliance of the external surface 111 of the first balloon 110 can be adopted.

In addition, when a mode is adopted in which the part of high compliance and the part of low compliance are provided in the external surface 111 of the first balloon 110 and further the compliance of the first balloon 110 and the compliance of the second balloon 120 are made different from each other, the compliance of the part of high compliance in the external surface 111 of the first balloon 110, the compliance of the part of low compliance in the external surface 111 of the first balloon 110, and the compliance of the second balloon 120 are set so as to be decreased in this order.

As a method of adjusting the compliance of the balloons 110 and 120, publicly known methods can be adopted as appropriate, such as, for example, as a method of adjusting the compliance of the balloons 110 and 120 by decreasing or increasing the material thicknesses of the balloons and a method of adjusting the compliance of the balloons 110 and 120 by adding a predetermined material to the balloons.

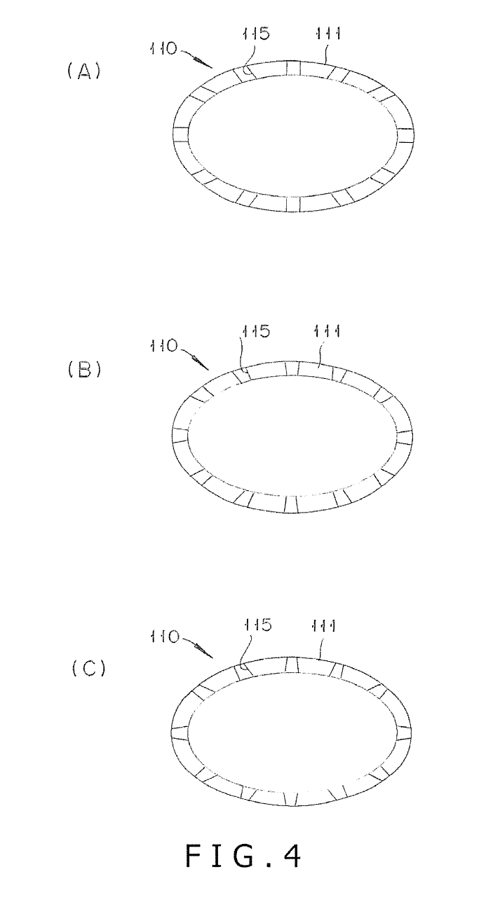

As shown in FIG. 2(A), FIG. 2(B), and FIG. 3, the first balloon 110 can be provided with through holes 115 that penetrate from the inside of the first balloon 110 to the external surface 111 of the first balloon 110. The through holes 115 function as a supply portion for supplying the adhesive material to the external surface 111 of the first balloon 110. That is, the adhesive material made to flow into the internal space 113 of the first balloon 110 is made to flow out to the external surface 111 of the first balloon 110 via the through holes 115, whereby the adhesiveness of the external surface 111 to the biological tissue t can be enhanced. The through holes 115 can be formed so as to have cross-sectional shapes as shown in FIGS. 4(A) to 4(C), for example.

As shown in FIG. 4(A), the through holes 115 can be formed so as to have a constant diameter from the internal space 113 of the first balloon 110 to the external surface 111. When the through holes 115 are formed in such a cross-sectional shape, outflows of the adhesive material flowing out from the respective through holes 115 can be made uniform, and therefore amounts of adhesion of the adhesive material on different parts of the external surface 111 of the first balloon 110 can be made uniform. In addition, as shown in FIG. 4(B), the through holes 115 can be formed in a tapered shape that increases in diameter from the internal space 113 of the first balloon 110 to the external surface 111. When the through holes 115 are formed in such a cross-sectional shape, the adhesive material is jetted out from the through holes 115 and sprayed on large areas of the biological tissue t. This increases an area where the biological tissue t and the external surface 111 of the first balloon 110 adhere to each other, so that the adhesiveness to the biological tissue t can be enhanced. In addition, as shown in FIG. 4(C), the through holes 115 can be formed in a tapered shape that decreases in diameter from the internal space 113 of the first balloon 110 to the external surface 111. When the through holes 115 are formed in such a cross-sectional shape, amounts of the adhesive material jetted out from the through holes 115 are reduced. Therefore, a jetting speed is increased. Thus, the flowing out of the adhesive material to the external surface 111 and the adhesion of the external surface 111 to the biological tissue t can be performed efficiently.

The cross-sectional shapes of the through holes 115 are not limited to the shapes illustrated in FIGS. 4(A) to 4(C), but can be changed as long as the adhesive material can be discharged from the internal space 113 of the first balloon 110 to the external surface 111. In addition, the number of through holes and the placement positions of the through holes are not limited to the number and the placement positions shown in the figures, but can be changed according to the mode of use of the balloon catheter.

As shown in FIG. 2(A) and FIG. 3, guide portions 117 for guiding flows of the adhesive material can be provided in the external surface 111 of the first balloon 110. The guide portions 117 can be formed by, for example, grooves communicating with the through holes 115 provided in the first balloon 110. The guide portions 117 distribute the adhesive material leaking out from the through holes 115 over the entire external surface 111 of the first balloon 110 along the guide portions 117, whereby the adhesiveness of the external surface 111 of the first balloon 110 to the biological tissue t is improved.

The shape, placement positions, and external shape of the grooves forming the guide portions 117 are not limited to the mode shown in the figures, but can be changed as long as the grooves have the function of guiding the flows of the adhesive material. For example, flow passages for forming the flows of the adhesive material utilizing capillarity can be constructed by forming the grooves whose width and depth are equal to or less than predetermined dimensions. In addition, for example, branch passages branching from one groove can be provided. By adopting such a constitution, the adhesive material can be distributed to the entire external surface 111 of the first balloon 110 efficiently.

As shown in FIG. 2(A) and FIG. 2(B), projecting portions 118 projecting toward the biological tissue t to which to adhere the external surface 111 of the first balloon 110 can be provided on the external surface 111. The projecting portions 118 are provided to increase the contact area where the external surface 111 of the first balloon 110 is in contact with the biological tissue t. The inner surface of pulmonary alveoli or the like included in the pulmonary parenchyma L does not have a smooth surface shape, but has a somewhat uneven surface shape, though there are individual differences (see FIG. 5). When the external surface 111 of the first balloon 110 is formed smoothly in contrast to such an inner surface shape, the contact area between the external surface 111 of the first balloon 110 and the biological tissue t cannot be increased. On the other hand, when the projecting portions 118 as shown in the figures are provided on the external surface 111 of the first balloon 110, the external surface 111 of the first balloon 110 can be brought into close contact with the inner surface of the biological tissue t, and therefore the contact area between the external surface 111 of the first balloon 110 and the biological tissue t can be increased.

As shown in FIG. 2(B) and FIG. 3, the balloon catheter 100 can be provided with connecting portions 160 for connecting the first balloon 110 and the second balloon 120 to each other. As shown in the figures, the connecting portions 160 can be formed by a column-shaped member that partially connects the internal surface of the first balloon 110 and an external surface 121 of the second balloon 120 to each other. However, the connecting portions 160 may be formed by partially bonding or fusing the first balloon 110 and the second balloon 120 with each other.

In a procedure using the balloon catheter 100, the adhesive material is supplied to the external surface 111 of the first balloon 110, and the external surface 111 of the first balloon 110 is adhered to the biological tissue t by the adhesive material. However, when the curing of the adhesive material present within the internal space 113 of the first balloon 110 progresses excessively before the first balloon 110 is contracted, the adhesive material supplied into the first balloon 110 cannot be discharged, so that the first balloon 110 cannot be contracted sufficiently. Even in such a case, when the first balloon 110 and the second balloon 120 are connected to each other via the connecting portions 160, the first balloon 110 can be contracted surely as the second balloon 120 is contracted. Further, when the connecting portions 160 are provided, the first balloon 110 can be expanded so as to be spread toward the biological tissue t as the second balloon 120 is expanded. Therefore, the contact area between the external surface 111 of the first balloon 110 and the biological tissue t can be increased by expanding the second balloon 120. In particular, when the compliance of the second balloon 120 is lower than the compliance of the first balloon 110, the expansion and contraction following properties of the first balloon 110 following the expansion and contraction of the second balloon 120 can be further improved.

The placement positions, placement number, and external shape, and the like of the connecting portions 160 are not limited to the mode shown in the figures, but can be changed as long as the first balloon 110 and the second balloon 120 are connected to each other so that the first balloon 110 can be contracted as the second balloon 120 is contracted. In addition, when the connecting portions are formed by various kinds of members, a metallic material, a resin material, and the like can be used as materials for the members.

As shown in FIG. 3, discharge portions 140 for discharging the adhesive material onto the external surface 111 of the first balloon 110 can be provided in the main body portion 130 of the balloon catheter 100. The discharge portions 140 function as a supply portion for supplying the adhesive material to the external surface 111 of the first balloon 110.

The discharge portions 140 can be formed by, for example, a branch passage 143 branching from the first lumen 131 provided in the main body portion 130 and an end opening 141 formed at an end of the branch passage 143, as shown in the figure. The discharge portions 140 discharge the adhesive material flowing through the first lumen 131 toward the external surface 111 of the first balloon 110 via the end openings 141. The discharged adhesive material is applied to the external surface 111 of the first balloon 110 to enhance the adhesiveness of the external surface 111 of the first balloon 110 to the biological tissue t. Incidentally, as shown in the figure, the end openings 141 of the discharge portions 140 can be arranged so as to face grooves forming guide portions 117. With such an arrangement, the discharged adhesive material can be made to flow into the grooves, and thus the adhesive material can be distributed over the entire external surface 111 of the first balloon 110 efficiently.

The placement positions and the like of the discharge portions 140 are not limited to the mode shown in the figure, but can be changed as long as the adhesive material can be discharged from the main body portion 130 of the balloon catheter 100 to the external surface 111 of the first balloon 110.

As shown in FIG. 3, the balloon catheter 100 can be provided with an attaching portion 171 for separably attaching the first balloon 110 to the main body portion 130 and an attaching portion 172 for separably attaching the second balloon 120 to the main body portion 130.

The attaching portions 171 and 172 can be formed by, for example, female screw portions 173a provided in the main body portion 130 and male screw portions 173b and 173c that are attached to the first balloon 110 and the second balloon 120, respectively, and which can be screwed into the female screw portions 173a, as shown in the figure. The first balloon 110 and the male screw portion 173b provided to the first balloon 110 are coupled to each other via a predetermined coupling member 174. In addition, the second balloon 120 and the male screw portion 173c provided to the second balloon 120 are coupled to each other via a predetermined coupling member 174. When the balloon catheter 100 is provided with the attaching portions 171 and 172, the balloons 110 and 120 can be detached from the main body portion 130 by simple work of rotating the main body portion 130 by a manual operation. Incidentally, the male screw portion 173b provided to the first balloon 110 and the male screw portion 173c provided to the second balloon 120 can be provided with a through hole not shown in the figure so as not to obstruct the flows of the fluids or the like into the internal spaces of the balloons.

The constitutions of the respective attaching portions 171 and 172 are not limited to the mode shown in the figure, but can be changed as long as the main body portion can be connected to and separated from the balloons. For example, structures, such as but not limited to, the following, may be used: a structure in which the connection and the separation are achieved by a fitting system; a structure in which the separation is achieved by pulling the main body portion 130 in a connected state; and a structure in which the separation is achieved by heating the connected parts.

A procedure using the balloon catheter 100 according to the present embodiment will next be described with reference to FIGS. 5 to 7. FIGS. 5 to 7 illustrate the steps from the introduction of the balloon catheter 100 into a pulmonary alveolus as an emphysematous pulmonary parenchyma L to decreasing of the volume of the pulmonary alveolus by the balloon catheter 100.

In performing the procedure, an operator identifies the pulmonary parenchyma L in an emphysematous and bulged state in advance by a preliminary examination or the like. Then, as shown in FIG. 5(A), the first balloon 110, the second balloon 120, and the main body portion 130 of the balloon catheter 100 are introduced into the pulmonary parenchyma L.

A bronchoscope generally used for the treatment of pulmonary emphysema, for example, can be used for the introduction of the balloon catheter 100. A bronchoscope is introduced into an airway p (a trachea, a main bronchus, a lobar bronchus, a bronchus, a bronchiole, and a terminal bronchiole or the like) so as to precede the balloon catheter 100. The balloon catheter 100 is inserted into a channel for catheter introduction which channel is provided in the bronchoscope, so that the balloon catheter 100 can be guided to a vicinity of the pulmonary parenchyma L. Incidentally, it is also possible to fold the first balloon 110 and the second balloon 120 in a predetermined shape before the introduction of the balloon catheter 100, and then introduce the balloon catheter 100 into the living body with the balloons 110 and 120 folded.

Next, a pressurizing medium is made to flow into the second balloon 120 to expand the second balloon 120. At this time, as shown in FIG. 5(B), the first balloon 110 located outside the second balloon 120 is also expanded with the expansion of the second balloon 120. Then, an adhesive material is supplied to the external surface 111 of the first balloon 110 in a state in which the external surface 111 of the first balloon 110 is in contact with the biological tissue t of the pulmonary parenchyma L. The external surface 111 of the first balloon 110 is thus adhered to the biological tissue t. Incidentally, in this step, the adhesive material may be supplied to the external surface 111 of the first balloon 110 while the pressurizing fluid is supplied to the second balloon 120.

Next, the pressurizing medium is discharged from the second balloon 120 to contract the second balloon 120. At this time, as shown in FIG. 6(A), the first balloon 110 is contracted as the second balloon 120 is contracted. When the first balloon 110 is contracted, the biological tissue t adhering to the external surface 111 of the first balloon 110 is pulled in the contracting direction of the first balloon 110. Incidentally, in this step, the adhesive material may be discharged from the first balloon 110 while the pressurizing medium is discharged from the second balloon 120.

As shown in FIG. 6(B), the biological tissue t is further contracted together with the first balloon 110 by continuing the contraction of the first balloon 110. As a result of the steps thus far, the volume of the diseased pulmonary parenchyma L can be decreased.

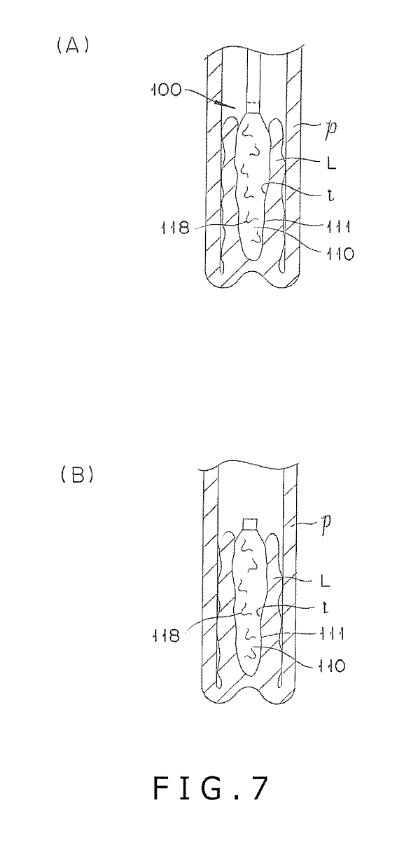

Thereafter, for example, as shown in FIG. 7(A), it is also possible to draw the balloon catheter 100 into the airway p, and thus draw the pulmonary parenchyma L into the airway p together with the balloon catheter 100. The volume of the diseased pulmonary parenchyma L can be further decreased by performing such a step. Then, as shown in FIG. 7(B), the main body portion 130 is detached from the first balloon 110 and the second balloon 120. Each of the balloons 110 and 120 is left indwelling in the living body, and the main body portion 130 is removed from the living body.

Incidentally, without the pulmonary parenchyma L being drawn into the airway p as shown in FIG. 7(A), the balloon catheter 100 may be taken out to the outside of the living body after the first balloon 110 and the biological tissue t are released from the adhering state. In addition, in order to prevent the pulmonary parenchyma L from swelling again after the pulmonary parenchyma L is contracted, a gel or an embolizing material in a fluidized state may be administered to the pulmonary parenchyma L.

The balloon catheter 100 according to the present embodiment can produce the following action and effect.

After the first balloon 110 provided to the balloon catheter 100 is expanded in living body, an adhesive material having fluidity is supplied to the external surface 111 of the first balloon 110 to adhere the external surface 111 to the biological tissue t of the diseased pulmonary parenchyma L. Further, the first balloon 110 is contracted, thereby the pulmonary parenchyma L can be contracted as the first balloon 110 is contracted. As a result, the volume of the diseased pulmonary parenchyma L can be decreased. According to the procedure using the balloon catheter 100, the pulmonary parenchyma can be physically contracted as the first balloon 110 is contracted. Thus, the puncturing operation performed in the bypass technique is rendered unnecessary. A minimally invasive procedure can therefore be realized. Further, no variations in treatment effect occur each time the procedure is performed. A stable treatment effect can therefore be obtained.

When the first balloon 110 is provided so as to be expandable by the injection of the adhesive material supplied through the first lumen 131, the expansion of the first balloon 110 and the adhesion of the external surface 111 of the first balloon 110 to the biological tissue t can be performed simultaneously. Thus, the procedure using the balloon catheter 100 can be performed more simply and more quickly.

In addition, when the balloon catheter 100 has the through holes 115 that penetrate from the inside of the first balloon 110 to the external surface 111 of the first balloon 110, the adhesive material flowing into the inside of the first balloon 110 can be made to flow out to the external surface 111 of the first balloon 110 via the through holes 115. Thus, the adhesiveness of the external surface 111 of the first balloon to the biological tissue t can be enhanced.

Further, when the balloon catheter 100 has the discharge portions 140 that discharge the adhesive material to the external surface 111 of the first balloon 110, the adhesive material can be directly applied to the external surface 111 of the first balloon 110. Thus, the adhesiveness of the external surface 111 of the first balloon 110 to the biological tissue t can be enhanced. The effect of treatment with the balloon catheter 100 can therefore be improved.

In addition, when at least a part of the external surface 111 of the first balloon 110 is formed so as to have higher compliance than other parts in the external surface 111, the part formed so as to have higher compliance in the external surface 111 can be partially deformed flexibly while the expandability of the first balloon 110 as a whole due to the introduction of the fluid is maintained. It is thereby possible to improve the contact property between the external surface 111 of the first balloon 110 and the biological tissue t, and thus increase the contact area between the external surface 111 of the first balloon 110 and the biological tissue t. The effect of treatment with the balloon catheter 100 can therefore be improved.

Additionally, when the balloon catheter 100 has the first balloon 110 and the second balloon 120 disposed inside the first balloon 110, the contacting of the first balloon 110 with the biological tissue and the drawing of the biological tissue t in the contracting direction of the first balloon 110 can be performed smoothly by controlling the expansion and contraction of the second balloon 120.

Furthermore, when the balloon catheter 100 has the connecting portions 160 that connect the first balloon 110 and the second balloon 120 to each other, the expansion and contraction following properties of the first balloon 110 following the expansion and contraction of the second balloon 120 can be improved. The effect of treatment with the balloon catheter 100 can therefore be improved.

When the compliance of the second balloon 120 is higher than the compliance of the first balloon 110, the external surface 111 of the first balloon 110 can be deformed flexibly so as to be along the inner surface shape of the biological tissue t when the first balloon 110 is expanded. Thus, the contact area where the external surface 111 of the first balloon 110 is in contact with the biological tissue t can be increased. The effect of treatment with the balloon catheter 100 can therefore be improved.

In addition, when the external surface 111 of the first balloon 110 is provided with the guide portions 117 that guide flows of the adhesive material, the guide portions 117 can distribute the adhesive material over the entire external surface 111 of the first balloon 110. Therefore, the adhesiveness of the external surface 111 of the first balloon 110 to the biological tissue t can be improved.

Furthermore, when the external surface 111 of the first balloon 110 is provided with the projecting portions 118 that project toward the biological tissue t, the external surface 111 of the first balloon 110 can be brought into close contact with the biological tissue t. Thus, the contact area between the external surface 111 of the first balloon 110 and the biological tissue t can be increased. The effect of treatment with the balloon catheter 100 can therefore be improved.

In addition, when the attaching portions 171 and 172 that separably attach the first balloon 110 and the second balloon 120, respectively, to the main body portion 130 are provided, a procedure for leaving the first balloon 110 and the second balloon 120 indwelling in the pulmonary parenchyma L can be performed. A degree of freedom of the procedure with the balloon catheter 100 can therefore be increased.

### Modifications

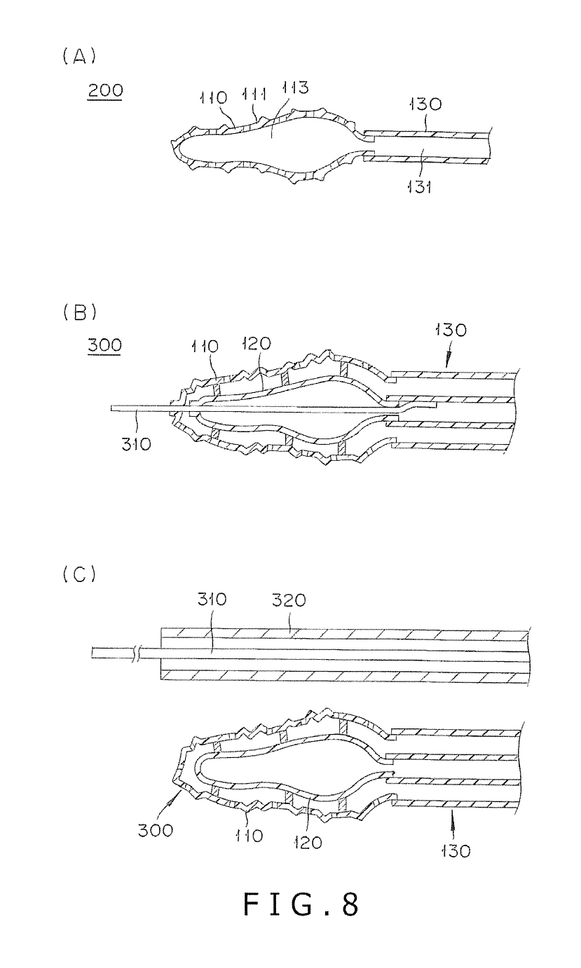

FIG. 8 is a diagram showing modifications of the foregoing embodiment. A first modification shown in FIG. 8(A) is a modification of the balloon structure of the balloon catheter. A second modification shown in FIG. 8(B) and a third modification shown in FIG. 8(C) are modifications of guide means for guiding the balloon catheter to the diseased pulmonary parenchyma. Incidentally, in the following description of the modifications, the same members as the members described in the foregoing embodiment will be identified by the same reference symbols, and description thereof will be omitted.

The first modification will first be described.

In the embodiment described earlier, the balloon catheter 100 is provided with the two balloons, that is, the first balloon 110 and the second balloon 120. However, a balloon catheter 200 is provided with only one balloon 110. An adhesive material is supplied to the external surface 111 of the balloon 110 provided to the balloon catheter 200, and the balloon 110 is contracted in a state in which the external surface 111 and a biological tissue t adhere to each other, whereby the volume of the pulmonary parenchyma L can be decreased. Incidentally, as shown in the figure, for example, the adhesive material may be supplied to the external surface 111 through through holes 115 that penetrate from the inside of the first balloon 110 to the external surface 111, and discharge portions 140 that function as a supply portion may be installed so that the adhesive material is supplied by the discharge portions 140. As for other constitutions, constitutions similar to those of the embodiment described earlier can be adopted.

The second modification and the third modification will next be described.

In the embodiment described earlier, a method has been illustrated in which the operation of introducing the balloon catheter 100 into the pulmonary parenchyma L is performed using a bronchoscope. However, a balloon catheter 300 can also be introduced into a living body by using a guide wire 310 generally used in the medical field, for example.

For example, as shown in FIG. 8(B), the operation of introducing the balloon catheter 300 can be performed by using the guide wire 310 integrally attached to the balloon catheter 300.

In addition, as shown in FIG. 8(C), a mode can also be adopted in which a balloon catheter 300 is introduced by using a guiding catheter 320 into which the balloon catheter 300 can be inserted together with a guide wire 310. When such a mode is adopted, a lumen for the insertion of the guide wire is separately provided to a main body portion 130.

The balloon catheter 300 can be introduced by the following procedure. The guide wire 310 is introduced to a predetermined treatment site by an endoscope or a bronchoscope. Further, the guiding catheter 320 is inserted along the guide wire 310. The guide wire 310 is thereafter extracted. The balloon catheter 300 is inserted into the guiding catheter 320 to place the balloon 110 at a target treatment site.

As shown in the second modification and the third modification described above, the method of introducing the balloon catheter into the pulmonary parenchyma as a treatment site can be changed as long as the balloon can be guided to a predetermined site.

The balloon catheter according to the present invention has been described above on the basis of embodiments and modifications thereof. However, the balloon catheter according to the present invention can be changed within the scope described in claims, and the constitution, material, structure, and the like of each member are not limited to only those described in the embodiments and the modifications described above.

1. A balloon catheter comprising:
a balloon, at least a part of an external surface of the balloon being brought into contact with a biological tissue when the balloon is expanded in living body;
a main body portion having a lumen through which a fluid for expanding the balloon can circulate; and
a supply portion for supplying the external surface of the balloon with an adhesive material, the adhesive material adhering the external surface of the balloon to the biological tissue.

2. The balloon catheter according to claim 1, wherein the balloon is provided so as to be expandable by injection of the adhesive material supplied through the lumen.

3. The balloon catheter according to claim 1, wherein the supply portion has a through hole penetrating from an inside of the balloon adhered to the biological tissue to the external surface of the balloon.

4. The balloon catheter according to claim 2, wherein the supply portion has a discharge portion provided in the main body portion, the discharge portion discharging the adhesive material to the external surface of the balloon adhered to the biological tissue.

5. The balloon catheter according to claim 1, wherein at least a part of the external surface of the balloon is formed so as to have higher compliance than other parts in the external surface.

6. The balloon catheter according to claim 1,
wherein the balloon includes
a first balloon having the external surface brought into contact with the biological tissue, the first balloon being expanded by injection of the adhesive material, and
a second balloon disposed such that at least a part of the second balloon is covered by the first balloon, the second balloon being expandable and contractible separately from the first balloon, and
the main body portion includes
a first lumen for circulating the adhesive material to and from the first balloon, and
a second lumen for circulating a fluid for expanding the second balloon to and from the second balloon.

7. The balloon catheter according to claim 6, further comprising a connecting portion for connecting the first balloon and the second balloon to each other.

8. The balloon catheter according to claim 6, wherein compliance of the first balloon is higher than compliance of the second balloon.

9. The balloon catheter according to claim 1, wherein the external surface of the balloon adhered to the biological tissue is provided with a guide portion for guiding a flow of the adhesive material.

10. The balloon catheter according to claim 9, wherein the external surface of the balloon adhered to the biological tissue is provided with a projecting portion projecting toward the biological tissue.

11. The balloon catheter according to claim 1, further comprising an attaching portion for separably attaching the balloon adhered to the biological tissue to the main body portion.

12. The balloon catheter according to claim 1, wherein the balloon is expandable and contractible.

13. The balloon catheter according to claim 1, wherein the adhesive material is fluid.

14. A balloon catheter, wherein the balloon comprises
a first balloon having the external surface brought into contact with the biological tissue, the first balloon being expanded by injection of the adhesive material, and
a second balloon disposed such that at least a part of the second balloon is covered by the first balloon, the second balloon being expandable and contractible separately from the first balloon, and
a main body portion, wherein the main body portion comprises
a first lumen for circulating the adhesive material to and from the first balloon, and
a second lumen for circulating a fluid for expanding the second balloon to and from the second balloon.
